(19) United States
(12) Patent Application Publication
(10) Pub. No.: US 2016/0213372 A1
(43) Pub. Date: Jul. 28, 2016

(54) A DEVICE AND METHOD FOR SUTURING HOLLOW ORGANS

(71) Applicant: KEREN MEDICAL LTD., JERUSALEM (IL)

(72) Inventors: Eliahu ELIACHAR, HAIFA (IL); Nir LILACH, KFAR YEHOSHUA (IL); Amir BARZILAY, KOKHAV YAIR (IL)

(73) Assignee: KEREN MEDICAL LTD., JERUSALEM (IL)

(21) Appl. No.: 15/025,404

(22) PCT Filed: Sep. 28, 2014
(86) PCT No.: PCT/IL2014/000048

§ 371 Date: Mar. 28, 2016

Related U.S. Application Data
(60) Provisional application No. 61/884,060, filed on Sep. 29, 2013.

(51) Int. Cl. A61B 17/11

(52) U.S. Cl. CPC A61B 17/11; A61B 2017/1103; A61B 2017/0472

(57) ABSTRACT

A new device for suturing a first hollow organ to a second hollow organ, comprising: a guiding-shaft, configured for temporary attachment to a fastenable-suture and guidance of the same via the tissues of the first and second hollow organs; and a foldable arm, configured to receive the guiding-shaft with the fastenable-suture. The device configured to manipulate the guiding-shaft and the foldable arm via the lumens of the first and second hollow organs, such that the fastenable-suture is guided and fastened to a tied-suture in a single act. According to a preferred embodiment, the device further comprises at least one additional guiding-shaft, for at least one additional fastenable-suture and at least one additional foldable arm; such that at least two independent tied-sutures are simultaneously delivered, from the lumens of the first and second hollow organs, in a single act.

### BACKGROUND OF THE INVENTION

Certain surgical operations require the removal of body parts; consequently other body parts must be reconnected in order to maintain normal body functions. For example, in certain heart operations where bypass surgery is performed, sections of a person's coronary artery to the heart may be either completely replaced or actually bypassed during the heart bypass operation. While some of these arteries are large and can be easily manipulated by a surgeon, other arteries or hollow body organs might be smaller and thus more difficult to manipulate due to their location within the body.

Surgical procedures tend to minimize an incision in a body tissue by utilizing advanced technology medical devices. Consequently, alternative suturing technologies and procedures have to be applied to properly address the growing complexity of intra-body suturing requirements. In the procedure of anastomosis of the urethra and the bladder during radical prostatectomy, for example, with the absence of suitable suturing technologies, the attachment of the urethral stump to the bladder neck is particularly difficult and to date carried out completely manually by the Urology surgeon.

The prior art of manual suturing cannot be applied in these surgical procedures without further abdominal cutting that provides an easier access to the tissues' area. This procedure has significantly high morbidity because of the presence of several large blood vessels in the operating region. It is not uncommon for the needles to slip or tear through these blood vessels and cause significant bleeding. Another disadvantage of the abdominal approach is the risk of punctures to the surgeon's finger; such punctures could lead to transmission of hepatitis, auto-immune deficiency Syndrome (AIDS) and other serious diseases.

Prior art inventions attempt to suture hollow organs include the following:

Amarasinghe, U.S. Pat. No. 4,553,543, disclosed a slotted, partially-flared, cylindrically shaped core mounted inside a tubularly-shaped sleeve with a flared end portion extending out the end of the sleeve. A flexible needle, which is longer than either the core or the sleeve, is held in each of the core slots by the sleeve, but is allowed freedom of longitudinal movement. Threads attached to the needles are inserted through a wall of a tubular body duct by inserting the flared end portion of the core into the body duct and then forcing the needles to move longitudinally in the slots against the flared end portion so that they bend radially outwardly through the walls of the body duct.

Orban III, U.S. Pat. No. 8,177,799, disclosed an apparatus and methods for performing a surgical anastomotic procedure. The apparatus include at least one fastener including a first fastener portion having an anchoring leg portion, a second fastener portion including an anchoring leg portion, wherein the first and second fastener portions are operatively associated with one another to selectively fix the position of the first fastener portion relative to the second fastener portion.

The above mentioned inventions disclose devices and methods for suturing hollow organs, where the actual fastening of the two hollow organs is either manually tied or just fastened by two wirely connected hooks.

Eliachar, US patent application No. 20110196393, disclosed in a former invention, a urethral catheter adapted for anastomosis following radical prostatectomy, comprising an inflated activating balloon adapted for concurrently (i) pressing the bladder neck to the urethra stub; (ii) effectively stretching suture; (iii) activating locks that non-reversibly catch the sutures; and (iv) cutting the distal portion thereof.

Thus, there is still a long felt need for an apparatus that can simplify the automatic suturing of the two hollow organs, from the lumen of the hollow organs, to a single and simple act.

### SUMMARY OF THE INVENTION

It is one object of the present invention to disclose a device for suturing a first hollow organ [610] to a second hollow organ [620], comprising:
a. a guiding-shaft [210], configured for temporary attachment to a first end of a fastenable-suture [110] and guidance of the same via the tissue of the first and second hollow organs [610,620]; and
b. a foldable arm [310], configured to accommodate the second end of the fastenable-suture and to receive the guiding-shaft [210] with the first end;
   wherein the device [100] configured to manipulate the guiding-shaft [210] and the foldable arm [310] via the lumens [615,625] of the first and second hollow organs [610,620], such that the fastenable-suture [110] is guided and fastened to form a tied-suture [510] in a single act.

It is another object of the present invention to disclose the device as defined above, wherein the device [100] further comprises:
a. at least one additional guiding-shaft [210], for at least one additional fastenable-suture [110]; and
b. at least one additional foldable arm [310];
   such that at least two independent tied-sutures [510] are simultaneously formed, from the lumens [615,625] of the first and second hollow organs [610,620], in the single act.

It is another object of the present invention to disclose the device as defined above, wherein the fastenable-suture [110] comprises:
a. a cord [115];
b. a male-head [120] having an anchor [121], at first end of the cord [115], configured for the temporary attachment with the guiding-shaft [210]; and
c. an accepting female-head [130], at opposite end of the cord [115], configured to receive the male-head [120] and form the tied-suture [510].

It is another object of the present invention to disclose the device as defined above, wherein the guiding-shaft [210] configured to guide the male-head [120] into the female-head [130], via the tissue of the first and second hollow organs [610,620], such that the tied-suture [510] is obtained.

It is another object of the present invention to disclose the device as defined above, wherein the foldable arm [310] comprises an open-housing [320] configured to temporarily accommodate the female-head [130] and release the same, upon retraction of the foldable arms [310].

It is another object of the present invention to disclose the device as defined above, wherein the device further comprising a longitudinal section [340]; the longitudinal section [340] configured to:
a. accommodate at least one the foldable arm [310];
b. accommodate at least one the guiding-shaft [210]; and
c. temporarily accommodate at least one the fastenable-suture [110].

It is another object of the present invention to disclose the device as defined above, wherein the foldable arm [310] configured to protrude inside the second hollow organ [620] and position the female-head [120] to receive the male-head [120].

It is another object of the present invention to disclose the device as defined above, wherein the foldable arm [310] configured to push and approximate the second hollow organ [620] towards the first hollow organ [610].

It is another object of the present invention to disclose the device as defined above, wherein the longitudinal section [340] configured to allow a reciprocal force between the guiding-shaft [210] and foldable arm [310], such that the tissues of first and second hollow organs [610,620] are pressed one against the other.

It is another object of the present invention to disclose the device as defined above, wherein the longitudinal section [340] further comprises at least one proximal guiding-element [410], configured to guide the guiding-shaft [210] into the female-head [130], via the tissues of the first and second hollow organs [610,620].

It is another object of the present invention to disclose the device as defined above, wherein the proximal guiding-element [410] comprises a shape selected from a group consisting of: U-shape, V-shape, circular-shape, elliptic-shape, polygonal-shape, and any combination thereof.

It is another object of the present invention to disclose the device as defined above, wherein the device [100] further comprises at least one distal guiding-element [420], configured to guide the guiding-shaft [210] into the female-head [130], via the tissues of the first and second hollow organs [610,620].

It is another object of the present invention to disclose the device as defined above, wherein the distal guiding-element [420] comprises a shape selected from a group consisting of: U-shape, V-shape, circular-shape, elliptic-shape, polygonal-shape, and any combination thereof.

It is another object of the present invention to disclose the device as defined above, wherein the distal guiding-element [420] is controlled by a horizontal slide [425] moving forward and backward, such that the distal guiding-element [420] is protruded and retracted accordingly.

It is another object of the present invention to disclose the device as defined above, wherein the foldable arm [310] comprises a foldable shield [330] configured to protect the second hollow organ [620] from undesired damage by the guiding-shaft [210];

It is another object of the present invention to disclose the device as defined above, wherein the shield [330] is at least partially flexible.

It is another object of the present invention to disclose the device as defined above, wherein the suture's cord [115] comprises a weakened section [116], which is thinner than the cord [115], configured to be ripped or lengthened to limit over tightening of the tied-suture [510].

It is another object of the present invention to disclose the device as defined above, wherein the guiding-shaft [210] comprises a disengaging-rod [215] threaded through the guiding-shaft [210], configured for relative motion inside the guiding-shaft [210]; the disengaging-rod [215] configured to disengage and detach the male-head [210] from the guiding-shaft [210], such that the anchor [121] of the tied suture [510] released and/or detached from the guiding-shaft [210].

It is another object of the present invention to disclose the device as defined above, wherein the device [100] configured for suturing of the first and second hollow organs [610,620] having orifices [616,626] with different diameters, shapes and/or volumes.

It is another object of the present invention to disclose the device as defined above, wherein the device [100] configured to penetrate into the first and second hollow organ [610,620] with or without puncturing the first and/or second hollow organs [610,620].

It is another object of the present invention to disclose the device as defined above, wherein of the longitudinal section [340] comprising a sharp or rounded head [345].

It is another object of the present invention to disclose the device as defined above, wherein the head [345] of the longitudinal section [340] is at least partially flexible.

It is another object of the present invention to disclose the device as defined above, wherein the cord [115] further comprising one or more protruding teeth [6].

It is another object of the present invention to disclose the device as defined above, wherein the fastenable-suture [110] is guided and fastened to form the tied-suture [510] in at least one additional act.

It is another object of the present invention to disclose the device as defined above, wherein the foldable arm [310] configured to be constantly deployed.

It is another object of the present invention to disclose a method for suturing a first hollow organ [610] to a second hollow organ [620], comprising steps of:
a. approximating the first and second hollow organs [610,620] one to another; and
b. suturing the first and second hollow organs [610,620] one to another, by means of forming at least one tied-suture [510];
   wherein the steps of approximating and suturing performed via the lumens [615,625] of the first and second hollow organs [610,620].

It is another object of the present invention to disclose the method as defined above, further comprising steps of:
a. penetrating into the first hollow organ [610], via an orifice [614] in the first hollow organ [610] or by puncturing the same;
b. penetrating into the second hollow organ [620], via an orifice [626] in the second hollow organ [620] or by puncturing the same; and
c. attaching the first and second hollow organs [610,620] one to another.

It is another object of the present invention to disclose the method as defined above, further comprising steps:
a. temporary attaching at least one fastenable-suture [110] to at least one guiding-shaft [210];
b. guiding the guiding-shaft [210] via the tissue of the first and second hollow organs [610,620]; and
c. receiving a the guiding-shaft [210] by at least one foldable arm [310];
   thereby fastening the fastenable-suture [110] and forming the at least one tied-suture [510] in at least one act.

It is another object of the present invention to disclose the method as defined above, further comprising step of providing the fastenable-suture [110] with:
a. a cord [115];
b. a male-head [120] having an anchor [121], at first end of the cord [115], for the temporarily attaching the guiding-shaft [210]; and
c. an accepting female-head [130], at opposite end of the cord [115], for receiving the male-head [120] and fastening the tied-suture [510].

It is another object of the present invention to disclose the method as defined above, wherein the step of suturing further comprising:
a. guiding the male-head [120] into the tissues of the first and second hollow organs [610,620], by the guiding-shaft [210]; and
b. accepting the male-head [120], by the female-head [130].

It is another object of the present invention to disclose the method as defined above, further comprising step of selecting at least one step from a group consisting of:
a. pushing the walls of the first hollow organ [610], by protruding the guiding-shaft [210];
b. protruding the foldable arm [310];
c. retracting the foldable arm [310];
d. folding the foldable arm [310];
e. pushing the second hollow organ [620] toward the first hollow organ [610];
f. fastening the tied-suture [510];
g. retracting the guiding-shaft [210].

It is another object of the present invention to disclose the method as defined above, further comprising step of selecting at least one step from a group consisting of:
a. disconnecting the female-head [120] from foldable arm [310], upon retraction of the foldable arm [310];
b. disconnecting the male-head [120] from the cord [115]; and
c. any combination thereof.

It is another object of the present invention to disclose the method as defined above, further comprising steps of temporarily accommodating the female-head [130] by an open-housing [320] within the foldable arm [310] and releasing the same, upon retracting of the foldable arm [210].

It is another object of the present invention to disclose the method as defined above, further comprising step of providing at least one proximal guiding-element [410], configured for guiding the guiding-shaft [210] into the female-head [130], via the first and second hollow organs [610,620].

It is another object of the present invention to disclose the method as defined above, further comprising step of providing at least one distal guiding-element [420], configured for guiding the guiding-shaft [210] into the female-head [130], via the first and second hollow organs [610,620].

It is another object of the present invention to disclose the method as defined above, further comprising step of selecting at least one step from a group consisting of controlling the distal guiding-element [420] with a horizontal slide [425], moving forward and backward, thereby protruding and retracting the distal guiding-element [420] accordingly.

It is another object of the present invention to disclose the method as defined above, further comprising step protecting the second hollow organ [620] from undesired damage by the guiding-shaft [210], by providing the foldable arm [310] with a foldable shield [330].

It is another object of the present invention to disclose the method as defined above, further comprising steps ripping and detaching the male-head [120] from the cord [115], when the cord's [115] tensile tension is reaching a predetermined tension force, by providing the suture's cord [115] with a weakened section [116].

It is another object of the present invention to disclose the method as defined above, further comprising steps of disengaging and separating the male-head [120] from the guiding-shaft [210], by providing the guiding-shaft [210] with a disengaging-rod [215] threaded through the center axis of the guiding-shaft [210] and configured for moving inside the guiding-shaft [210], thereby releasing the fastenable-suture [110] from the guiding-shaft [210].

It is still an object of the present invention to disclose a device [100] for suturing a first hollow organ [610] to a second hollow organ [620], comprising:
a. a guiding-shaft [210], configured for temporary attachment to a first end of a fastenable-suture [110] and guidance of the same via the tissue of the first and second hollow organs [610,620]; and
b. an arm, configured to accommodate the second end of the fastenable-suture [110] and to receive the guiding-shaft [210] with the first end;
   wherein the device [100] configured to manipulate the guiding-shaft [210] and the arm via the lumens [615,625] of the first and second hollow organs [610,620], such that the fastenable-suture [110] is guided and fastened to form a tied-suture [510] in a single act.

It is lastly an object of the present invention to disclose the wherein the device [100] further comprises:
a. at least one additional guiding-shaft [210], for at least one additional fastenable-suture [110]; and
b. at least one additional arm;
   such that at least two independent tied-sutures [510] are simultaneously formed, from the lumens [615,625] of the first and second hollow organs [610,620], in the single act.

### BRIEF DESCRIPTION OF THE DRAWINGS

The invention is herein described, by way of example only, with reference to the accompanying drawings, wherein:

FIG. 1 is a schematic drawing of the device for suturing a first hollow organ to a second hollow organ, in its closed arrangement;

FIG. 2 is a schematic drawing of the device in its closed arrangement, penetrating the lumen of the urethra;

FIG. 3 is a schematic drawing of the device inside the lumen of the urethra, where the guiding-shafts are slightly protruded;

FIG. 4 is a schematic drawing of the device, further penetrating into the lumen of the bladder neck;

FIG. 5 is a schematic drawing of the device in its open or deployed arrangement, where the foldable arms are protruding inside the lumen of the bladder neck;

FIG. 6 is a schematic drawing of the device in its open or deployed arrangement, where the foldable arms are protruded inside the lumen of bladder neck and the guiding-shafts push the fastenable-sutures;

FIG. 7 is a schematic drawing of the device in its open or deployed arrangement, where the foldable arms are protruded inside the lumen of the bladder neck and the guiding-shafts fasten the tied-sutures;

FIG. 8 is a schematic drawing of the device in its open or deployed arrangement, where the guiding-shafts detach the tied-sutures;

FIG. 9 is a schematic drawing of the device in its open or deployed arrangement, where the guiding-shafts are retracting from the foldable arms;

FIG. 10 is a schematic drawing of the device in its closed arrangement, after the foldable arms a retracted back into the device's longitudinal section;

FIG. 11 is a schematic drawing of the device in its closed arrangement, after the device has retracted out of the lumens of the first and second hollow organs;

FIGS. 12A and 12B are schematic cut-views of the first and second hollow organs, demonstrating the resulted tied-sutures;

FIG. 13 is a schematic cut-view of the device's longitudinal section, disclosing a proximal guiding element and a distal guiding element configured for the guidance of the guiding-shafts;

FIGS. 14A, 14B and 14C are schematic drawing disclosing the device's longitudinal section having head [345] which is flexible.

FIGS. 15 and 16 are a schematic prior art drawing of the fastenable-suture and the guiding-shaft, describing the various components of same;

FIGS. 17A, 17B, 17C, 17D and 17E are prior art schematic drawings of the fastenable-suture, comprising the accepting channels, being pushed by the guiding-shaft, shown in four stages: approaching, entering, fastening, retracting and detaching, respectively; and

FIG. 18 is a prior art schematic drawing the fastenable-suture, where the cord comprises a weakened section, configured to be ripped and facilitate the detachment of the male-head from the cord.

### DETAILED DESCRIPTION OF THE SPECIFIC EMBODIMENTS

The following description is provided, alongside all chapters of the present invention, so as to enable any person skilled in the art to make use of the invention and sets forth the best modes contemplated by the inventors of carrying out this invention. Various modifications, however, are adapted to remain apparent to those skilled in the art, since the generic principles of the present invention have been defined specifically to provide a device for suturing a first hollow organ to a second hollow organ via their lumens, such that the suturing is performed in a single act.

The term “hollow organ” refers herein to a visceral organ that is a hollow tube or pouch, such as the stomach, urinary bladder, urethra, intestine, an artery, a vein, or that includes a cavity, like the heart or the lung.

The term “lumen” refers herein to the internal space of a hollow organ with a tubular or pouch structure.

The term “over tightening” of a suture refers herein to tissue damage, rapture or any vascular occlusion.

The term “foldable arm” refers herein to an arm configured for reversible deployment and withdrawal by means of reversible folding, collapsing, retracting, and/or telescopic motion.

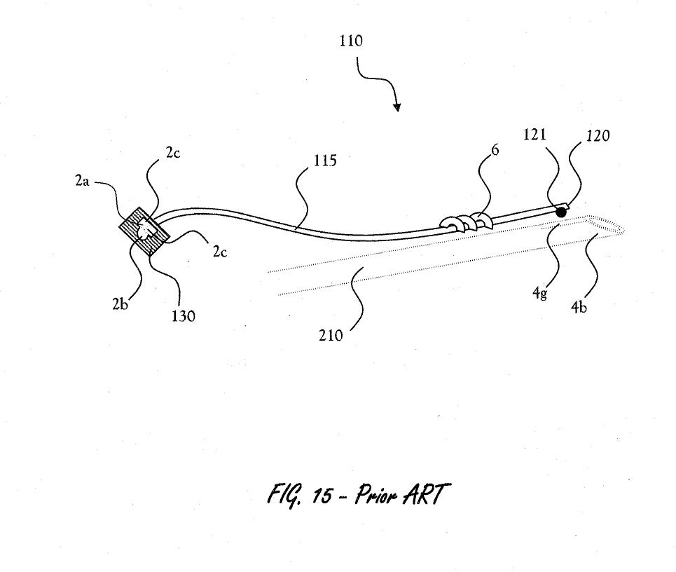

The term “fastenable-suture” [110] refers herein to a fastenable-suture [110] disclosed in US20110196393, which is incorporated herein as a reference, See e.g., FIGS. 15-18. Fastenable-suture [110] is provided useful for suturing a first tissue with a second tissue, and characterized by an elongated flexible cord [115], with one or more teeth [6] protruding from same.

The fastenable-suture [110] includes a male-head [120] at the distal end and an accepting female-head [130] at the proximal end. The male-head [120] is in connection with an anchor [121], where the anchor [121] is configured to be temporarily accommodated by a distal end [4b] of a guiding-shaft [210]. The female-head [130] includes a suture accepting-channel [2a] and a neighboring guiding-shaft accepting-channel [2b]. At least one of the two accepting-channels [2a, 2b] comprise one or more flexible pawls [2c] for both allowing, by means of the teeth [6], a continuous and linear forward motion of the male-head [120] and the cord [115] within the accepting-channel [2a, 2b] in only one direction, while preventing motion of the male-head [120] and cord [115] in a backward direction and allowing only the guiding-shaft [210] a reversible reciprocal motion.

The present invention is a new device [100] for suturing a first hollow organ [610] to a second hollow organ [620], comprising: a guiding-shaft [210], configured for temporary attachment to a fastenable-suture [110] and guidance of the same via the tissues of the first and second hollow organs [610,620]; and a foldable arm [310], configured to receive the guiding-shaft [210] with the fastenable-suture [110]; wherein the device [100] configured to manipulate the guiding-shaft [210] and the foldable arm [310] via the lumens [615,625] of the first and second hollow organs [610,620], such that the fastenable-suture [110] is guided and fastened to a tied-suture [510] in a single act.

The device [100], according to a preferred embodiment, further comprises at least one additional guiding-shaft [210], for at least one additional fastenable-suture [110]; and at least one additional foldable arm [310]; such that at least two independent tied-sutures [510] are simultaneously delivered, from the lumens [615,625] of the first and second hollow organs [610,620], in a single act.

According to another embodiment, the present invention is a new device [100] for suturing a first hollow organ [610] to a second hollow organ [620], comprising: a guiding-shaft [210], configured for temporary attachment to a fastenable-suture [110] and guidance of the same via the tissues of the first and second hollow organs [610,620]; and a retractable-arm [310], configured to receive the guiding-shaft [210] with the fastenable-suture [110]; wherein the device [100] configured to manipulate the guiding-shaft [210] and the retractable-arm [310] via the lumens [615,625] of the first and second hollow organs [610,620], such that the fastenable-suture [110] is guided and fastened to a tied-suture [510] in a single act.

The device [100], according to a preferred embodiment, further comprises at least one additional guiding-shaft [210], for at least one additional fastenable-suture [110]; and at least one additional retractable-arm [310]; such that at least two independent tied-sutures [510] are simultaneously delivered, from the lumens [615,625] of the first and second hollow organs [610,620], in a single act.

According to another embodiment, the present invention is a new device [100] for suturing a first hollow organ [610] to a second hollow organ [620], comprising: a guiding-shaft [210], configured for temporary attachment to a fastenable-suture [110] and guidance of the same via the tissues of the first and second hollow organs [610,620]; and an arm, configured to receive the guiding-shaft [210] with the fastenable-suture [110]; wherein the device [100] configured to manipulate the guiding-shaft [210] and the arm via the lumens [615,625] of the first and second hollow organs [610,620], such that the fastenable-suture [110] is guided and fastened to a tied-suture [510] in a single act.

The device [100], according to a preferred embodiment, further comprises at least one additional guiding-shaft [210], for at least one additional fastenable-suture [110]; and at least one additional arm; such that at least two independent tied-sutures [510] are simultaneously delivered, from the lumens [615,625] of the first and second hollow organs [610,620], in a single act.

According to another embodiment of the present invention the fastenable-suture [110] is guided and fastened to form the tied-suture [510] in at least one additional act.

The present invention is a new method for suturing a first hollow organ [620] to a second hollow organ [620], comprising steps of approximating the first and second hollow organs [610,620] one to another; and suturing the first and second hollow organs [610,620] one to another, by means of providing a tied-suture [510]; wherein the steps of approximating and suturing performed from the lumens [615,625] of the first and second hollow organs [610,620].

The method, according a preferred embodiment, further comprising steps of penetrating into the first hollow organ [610], via an orifice [614] in the first hollow organ [610] or by puncturing the same; penetrating into the second hollow organ [620], via an orifice [626] in the second hollow organ [620] or by puncturing the same; and attaching the first and second hollow organs [610,620] one to another.

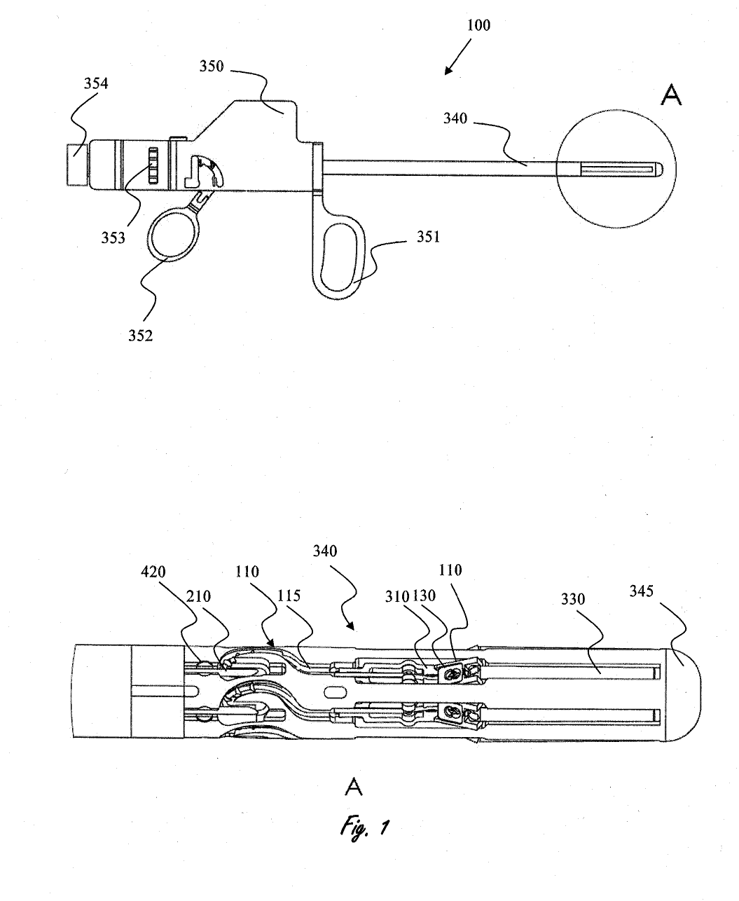

Reference is now made to FIGS. 1-11, which demonstrate the various configurations and positions of the disclosed device [100].

FIG. 1 is a schematic drawing of the device [100], in its closed arrangement, before its use. Two views are demonstrated: a whole device [100] view demonstrating the device's [100] gun-like section [350] and its longitudinal section [340]; and a close view (A) of the longitudinal section [340] and its various components.

The close view (A) discloses the device's [100] longitudinal section [340] configured to accommodate the at least one foldable arm [310] and its accompanying foldable shield [330]; accommodate the at least one guiding-shaft [210]; and temporarily accommodate the at least one fastenable-suture [110].

The close view (A) further discloses the arrangement of the fastenable-suture [110], its cord [115], and its female-head [130] accommodated by the longitudinal section [340] of the device [100].

The close view (A) further discloses the head [340] of the longitudinal section [345] which is either sharp (not shown) or rounded configured to penetrate into the first and second hollow organ [610,620] with or without puncturing the first and/or second hollow organs [610,620].

The close view (A) further discloses another embodiment of the present invention, where the device [100] further comprises at least one distal guiding-element [420], configured to guide the guiding-shaft [210] into the female-head [130], via the tissues of the first and second hollow organs [610,620]. The distal guiding-element [420], according to another embodiment of the present invention, comprises a shape selected from a group consisting of: U-shape, V-shape, circular-shape, elliptic-shape, polygonal-shape, and any combination thereof.

The whole device [100] view, as in FIG. 1, discloses the devices [100] gun-like section [350] comprising: a fixed handle [350], a sliding handle [352], a distal rolling element [353] and a proximal rolling element [354] all configured to manipulate the guiding-shafts [210] and the foldable arms [310] via the lumens [615,625] of the first and second hollow organs [610,620], such that the fastenable-suture [110] is guided and fastened to a tied-suture [510], in a single act.

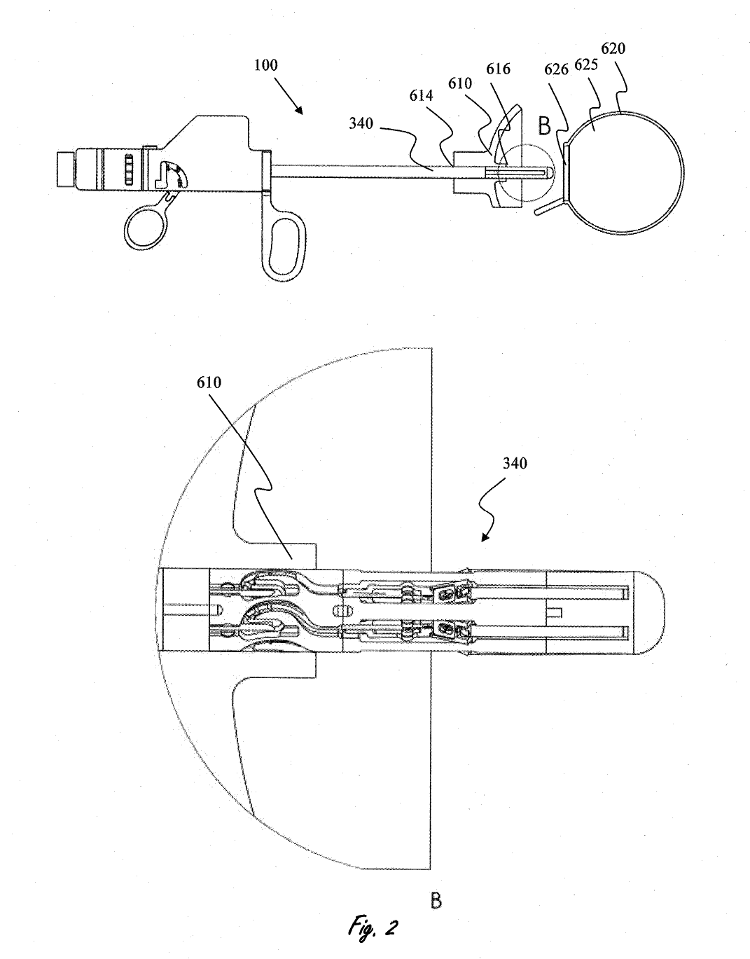

FIG. 2 is a schematic drawing of the device [100] in its closed arrangement, penetrating the first hollow organ [610], e.g. the shown urethra, via an orifice [614] and before penetrating into the second hollow organ [620], e.g. the shown bladder neck. Two views are demonstrated in FIG. 2: a whole device [100] view and a close view (B) of the longitudinal section [340] and its various components.

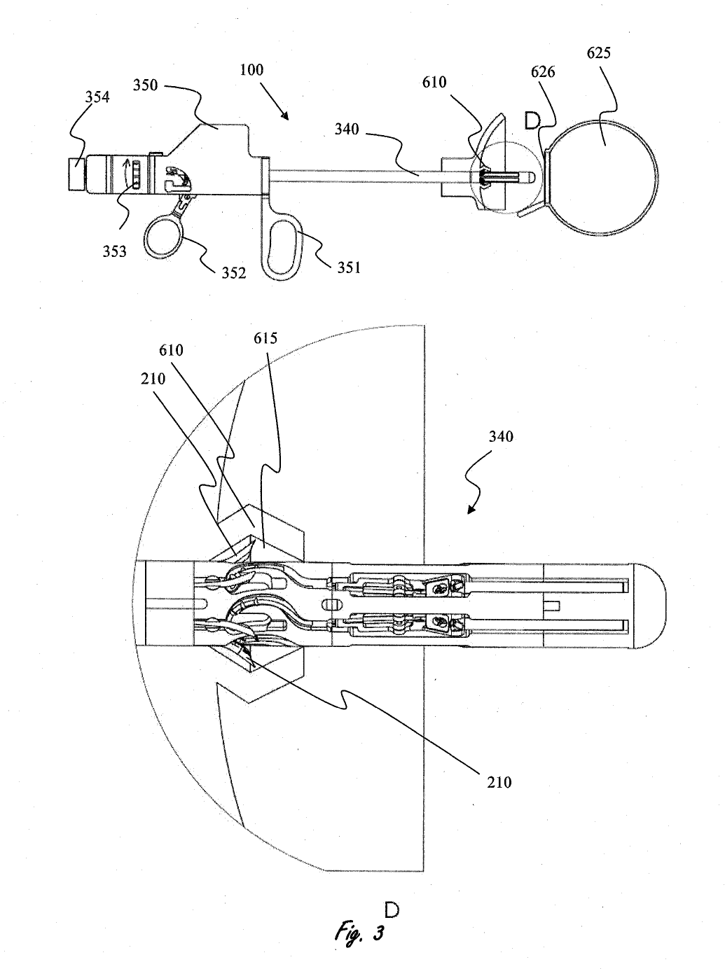

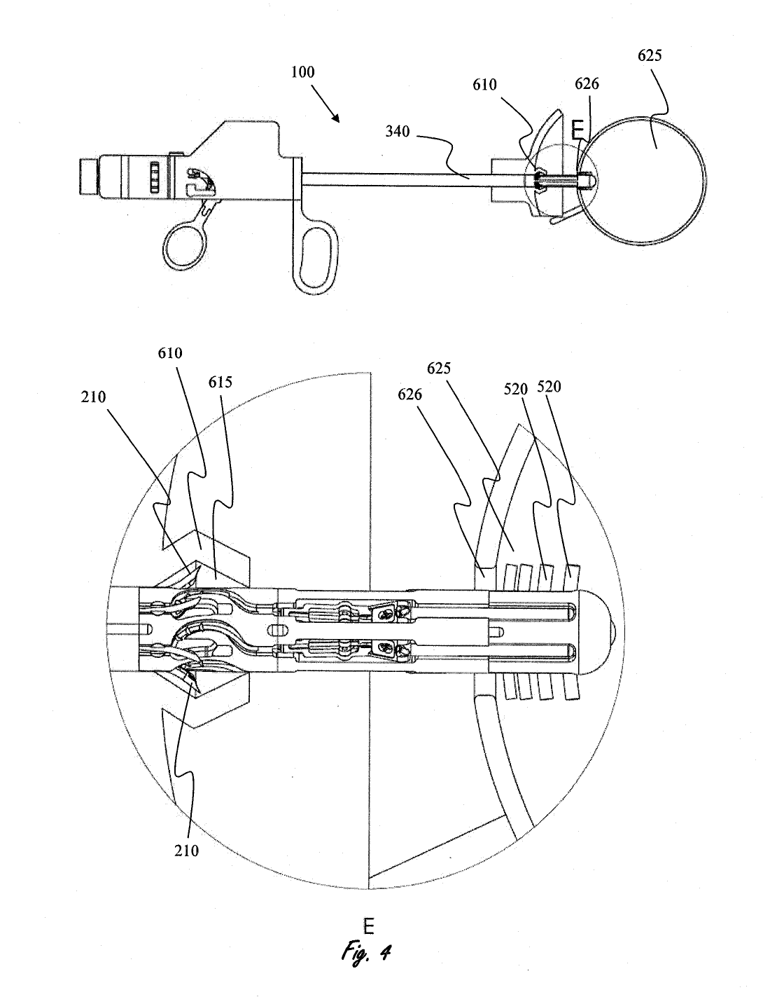

FIG. 3 is a schematic drawing of the device [100] inside the first hollow organ [610], e.g. the urethra, where guiding-shafts [210] are slightly protruded by protruding the distal guiding elements [420]. This procedure of protruding the guiding-shafts [210] and pushing the walls of the first hollow organ [610] is performed for marking the location of the device [100] within the lumen [615] of the first hollow organ [610] (also demonstrated in FIGS. 4 and 5), for assisting to the performing physician. Two views are demonstrated in FIG. 3: a whole device [100] view and a close view (D) of the longitudinal section [340] and its various components. It is further demonstrated, as in FIG. 3 whole device [100] view, that protruding of the distal guiding elements [420] is achieved by rolling the device's distal rolling element [353].

FIG. 4 is a schematic drawing of the device [100] further penetrating into the second hollow organ [620], e.g. the bladder neck, via its orifice [626]. Two views are demonstrated in FIG. 4: a whole device [100] view and a close view (E) of the longitudinal section [340] and its various components.

According to an embodiment of the present invention, the method for suturing further comprising a step of manually suturing and refashioning the orifice [616] of the first hollow organ [610] and/or the orifice [626] of the second hollow organ [626], thereby the providing the orifices [616,626] with about same size and/or shape. The close view (E), as in FIG. 4, further discloses an example of such suturing and refashioning of the bladder neck's orifice [626], resulting with at least one manual-suture [520].

FIG. 5 is a schematic drawing of the device [100] in its open or deployed arrangement, where the foldable arms [310] and their accompanying foldable shields [330] are protruding inside the second hollow organ [620], e.g. the bladder neck. Two views are demonstrated in FIG. 5: a whole device [100] view and a close view (G) of the longitudinal section [340] inside the first and second hollow organs [610,620]. It is further demonstrated, as in FIG. 5 whole device [100] view, that manipulation of the foldable arms [310] is achieved by rolling the proximal rolling element [354].

According to another embodiment of the present invention the foldable arm [310] is configured to be constantly deployed.

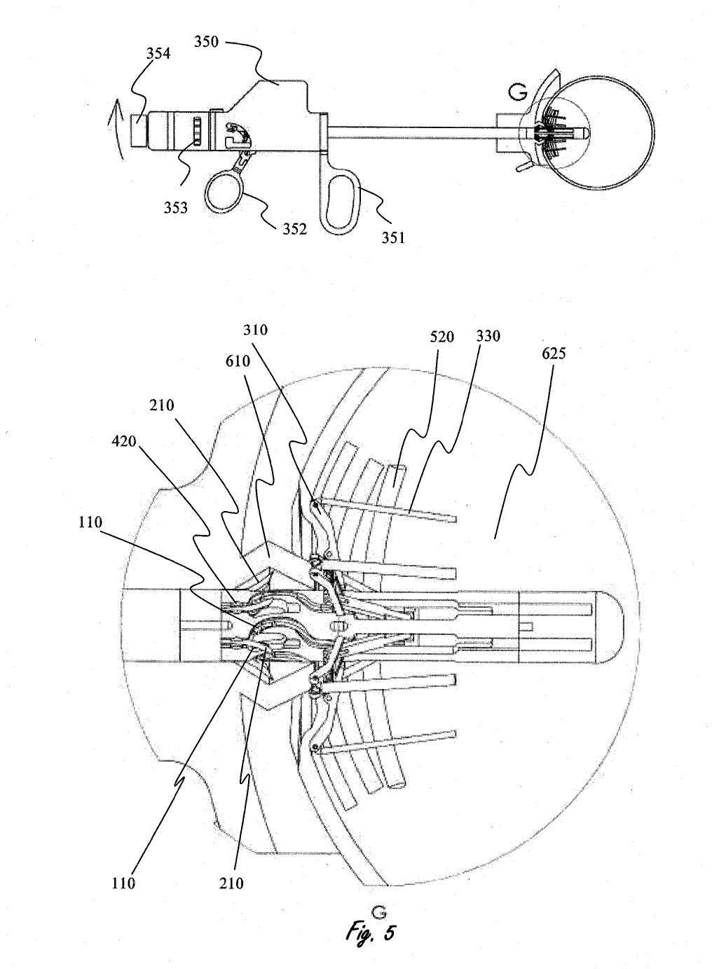

As demonstrated in FIG. 5 and in FIG. 6 the foldable arm [310] are configured, according a preferred embodiment, to push and approximate the second hollow organ [620] towards the first hollow organ [610], up until the first and second hollow organs [610,620] are presses against each other, as shown in FIG. 6. According to preferred embodiment the treated patient is in a Trendelenburg position, where the body is laid flat on the back (supine position) with the feet higher than the head by 15-30 degrees; this is a standard position used in anastomosis following a prostatectomy surgery, where the gravity assist in approximating the bladder neck to the urethra.

FIG. 6 is a schematic drawing of the device [100] in its open or deployed arrangement, where the foldable arms [310] are protruding inside the second hollow organ [620] e.g. bladder neck and the guiding-shafts [210] push the fastenable-sutures [110] male-head [120] into the female-head [130]. Two views are demonstrated in FIG. 6: a whole device [100] view and a close view (H) of the longitudinal section [340] inside the first and second hollow organs [610,620].

The close view (H) further demonstrates a preferred embodiment of the present invention where the foldable shields [330] are configured to protect the second hollow organ [620], e.g. the bladder neck, from undesired damage that may be conducted by the guiding-shafts [210].

It is further demonstrated, as in FIG. 6 whole device [100] view, that manipulation of the guiding-shafts [210] is achieved by sliding the sliding handle [352].

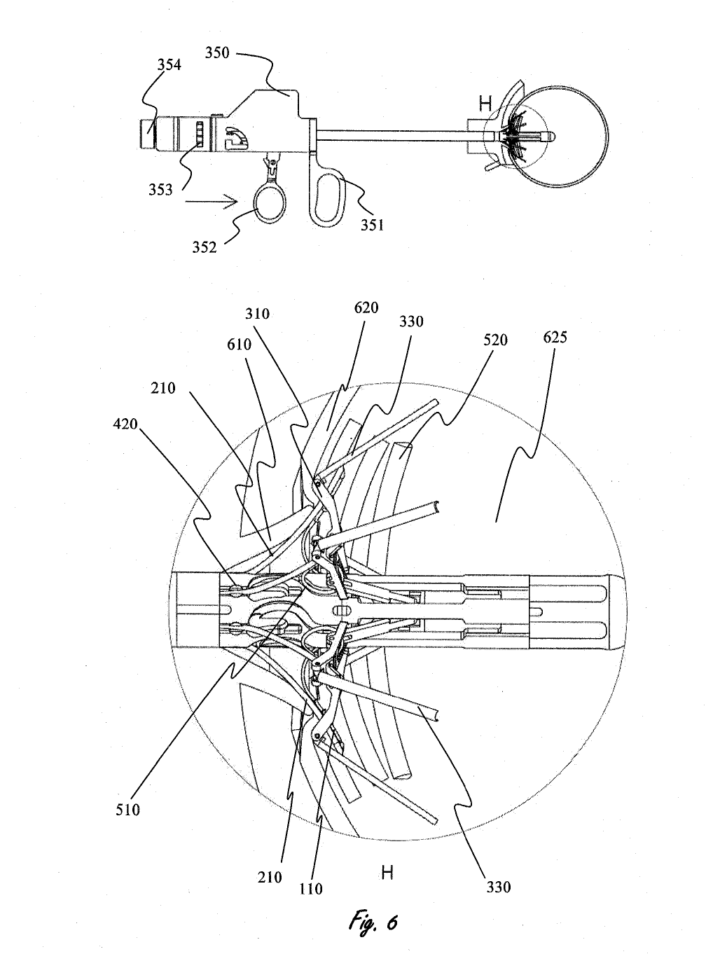

FIG. 7 is a schematic drawing of the device [100] in its open or deployed arrangement, where the foldable arms [310] are protruding inside the second hollow organ [620], e.g. the bladder neck, and where the guiding-shafts [210] fasten the tied-sutures [510]. Two views are demonstrated in FIG. 7: a whole device [100] view and a close view (I) of the longitudinal section [340] inside the first and second hollow organs [610,620].

According to preferred embodiment the suture's cord [115] comprises a weakened section [116], which is thinner than the cord [115] (as shown in FIG. 18), configured to be ripped and facilitate detachment of the male-head [120] from the cord [115], when the cord's [115] tensile tension reaches a predetermined tension force.

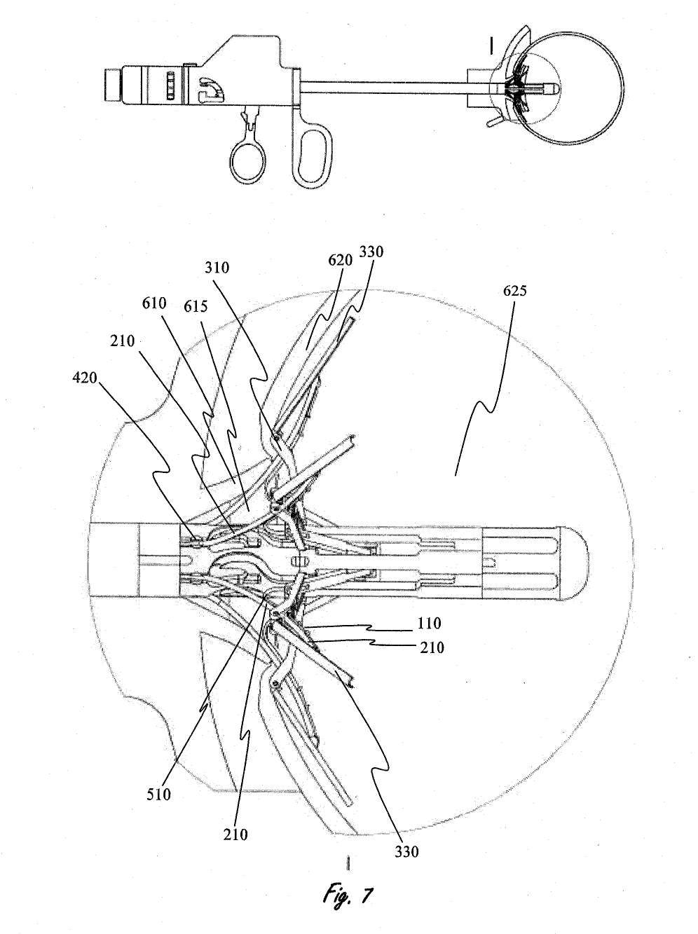

FIG. 8 is a schematic drawing of the device [100] in its open or deployed arrangement, where the guiding-shafts [210] detach from the tied-sutures [510], by means that the guiding-shafts [210] keep pushing the fastenable-sutures until the weakened section [116] of the cord [115] is ripped and the male-head [120] is detached from the cord [115]. Two views are demonstrated in FIG. 8: a whole device [100] view and a close view (J) of the longitudinal section [340] inside the first and second hollow organs [610,620]. It should be emphasized that each of tied-sutures [510] is detached according to the tensile tension created at its fastenable-suture [110], thereby the detaching of the various tied-sutures [510] is not necessarily simultaneous.

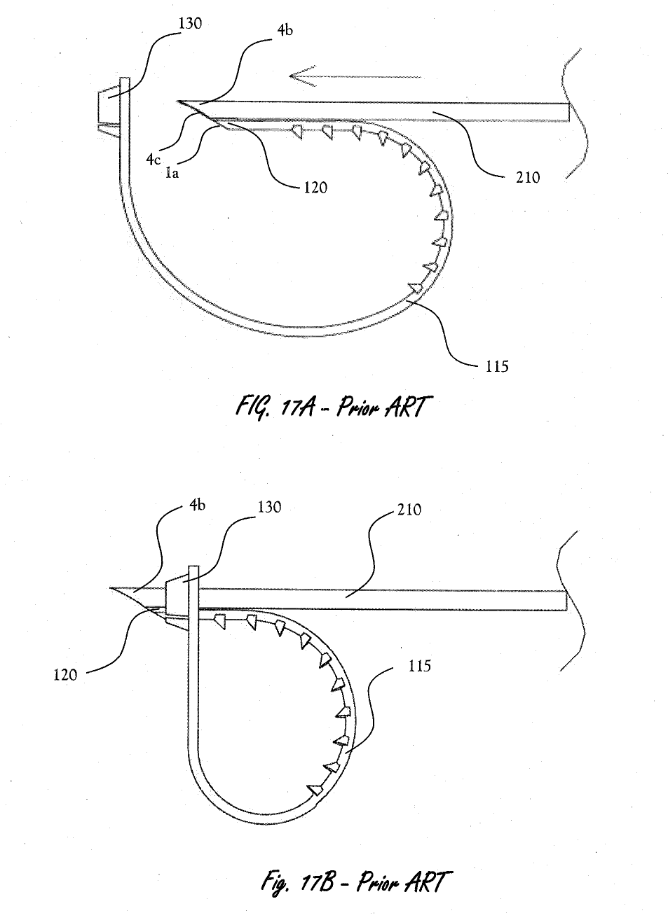

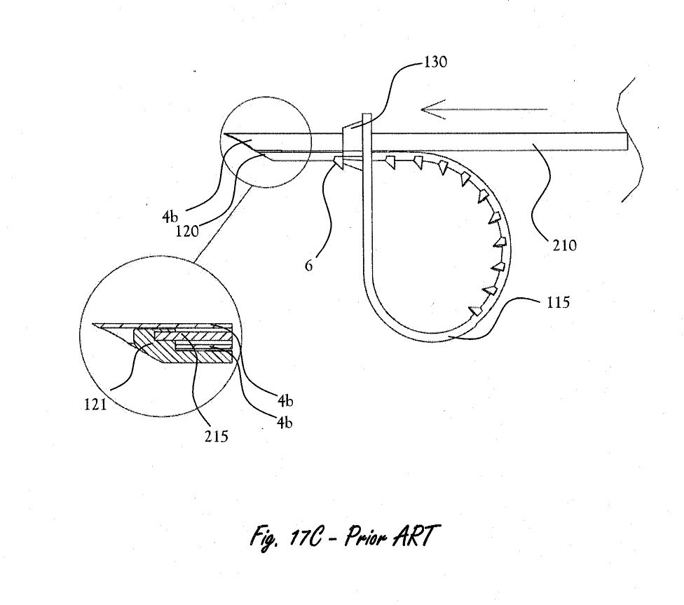

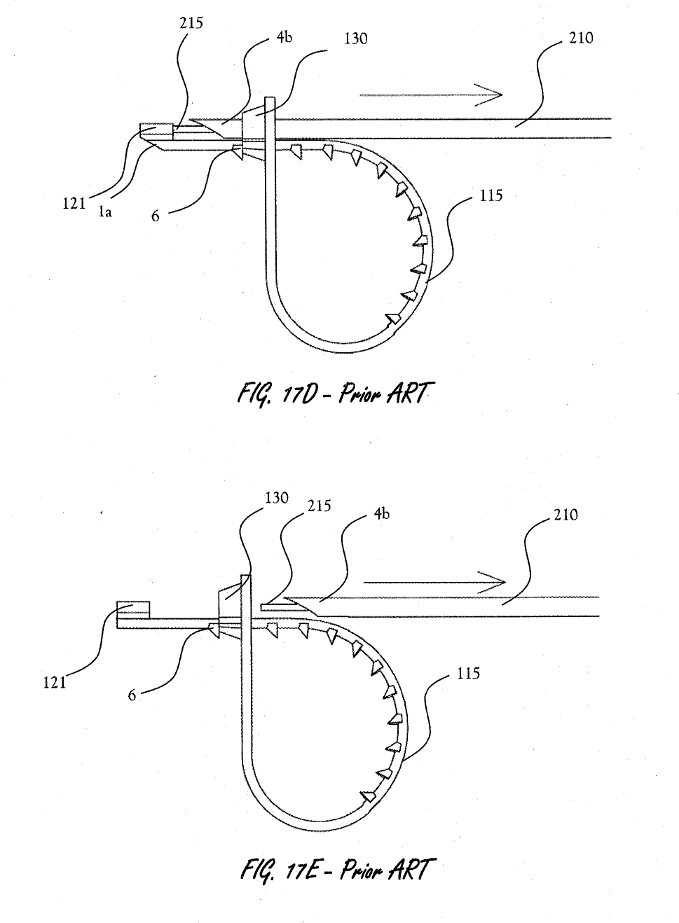

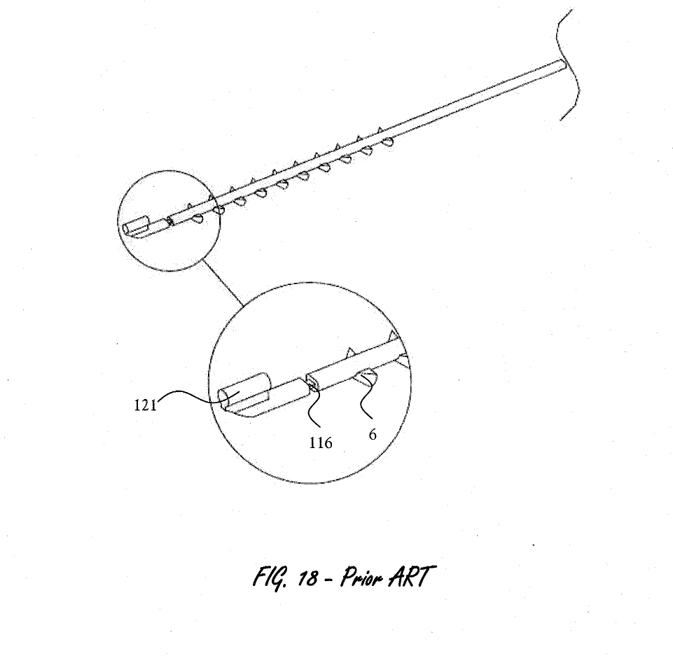

According to preferred embodiment the guiding-shaft [210] further comprises a disengaging-rod [215] threaded through the central axis of the guiding-shaft [210] (as in FIGS. 17C, 17D and 17E), configured for relative motion inside the guiding-shaft [210] in order to disengage the anchor [121] out of the guiding-shaft's [210], when the guiding-shaft [210] is retracting backward.

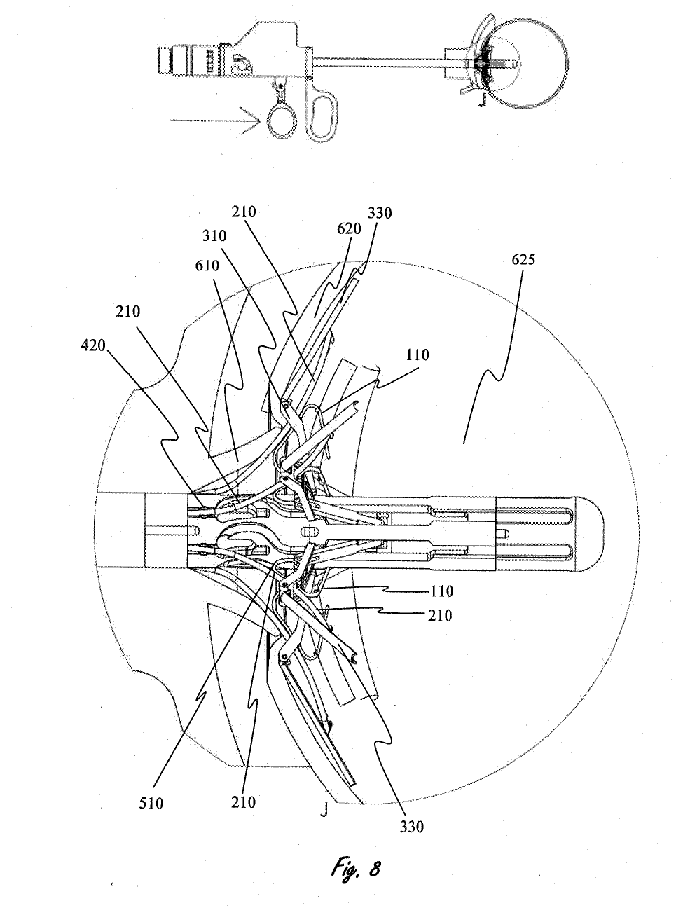

FIG. 9 is a schematic drawing of the device [100] in its open or deployed arrangement, where the guiding-shafts [210] are retracting from the foldable arms [310]. Two views are demonstrated in FIG. 9: a whole device [100] view and a close view (L) of the longitudinal section [340] inside the first and second hollow organs [610,620]. As shown in the close view (L), by retracting the guiding-shafts [210] the disengaging-rods [215] (as in FIG. 17C, 17D and 17E) disengage the anchors [121] out of the guiding-shafts [210], and the detached male-heads [120] fall away from the guiding-shafts [210].

FIG. 10 is a schematic drawing of the device [100] back in its closed arrangement, after the foldable arms [310] are retracted back into the device's longitudinal section [340]. Two views are demonstrated in FIG. 10: a whole device [100] view and a close view (M) of the longitudinal section [340] inside the first and second hollow organs [610,620]. As shown in the close view (M), the guiding-shafts [210], the foldable arms [310] and their accompanying foldable shield [330] are back at their original position, accommodated by the longitudinal section [340]. It further demonstrated that the female-heads [130] are released out of their accommodating open-housing [320]. The open-housing [320] are configured to temporarily accommodate the female-head [130] and release the same, upon retraction of the foldable arms [210]. According to a preferred embodiment, by retracting foldable arms [310] the pulling force required for releasing the female-head [130] is much smaller than the cords [115] tensile tension.

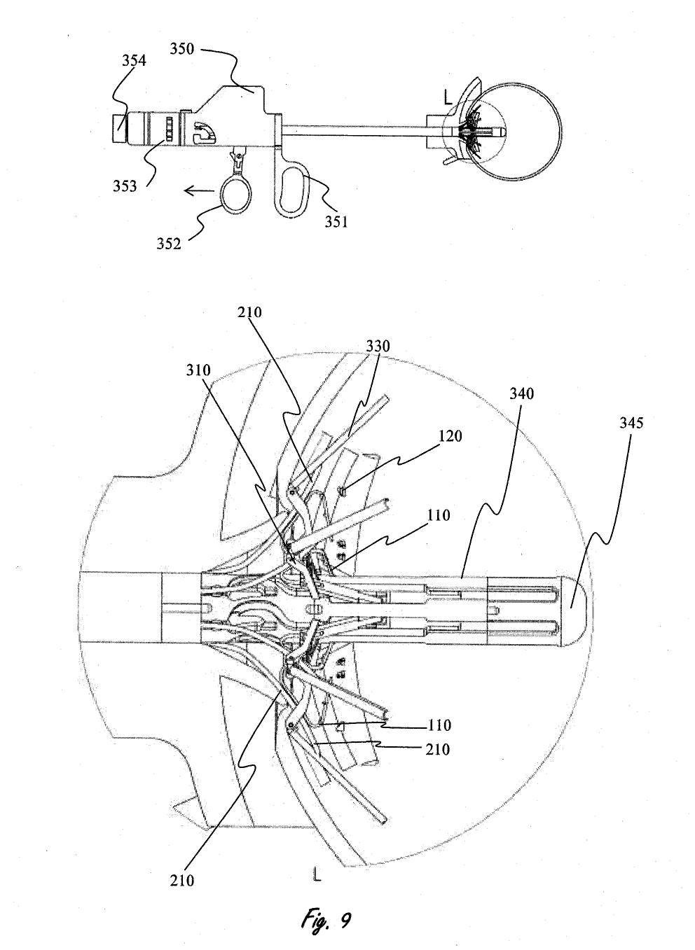

FIG. 11 is a schematic drawing of the device [100] in its closed arrangement, after the device [100] has retracted out of the first and second hollow organs [610,620]. Two views are demonstrated in FIG. 11: a whole device [100] view and a close view (N) of the longitudinal section [340] outside the first and second hollow organs [610,620]. As shown in the close view (N), the first and second hollow organs [610, 620] are sutured to one another, while allowing a flow between their lumens [615,625].

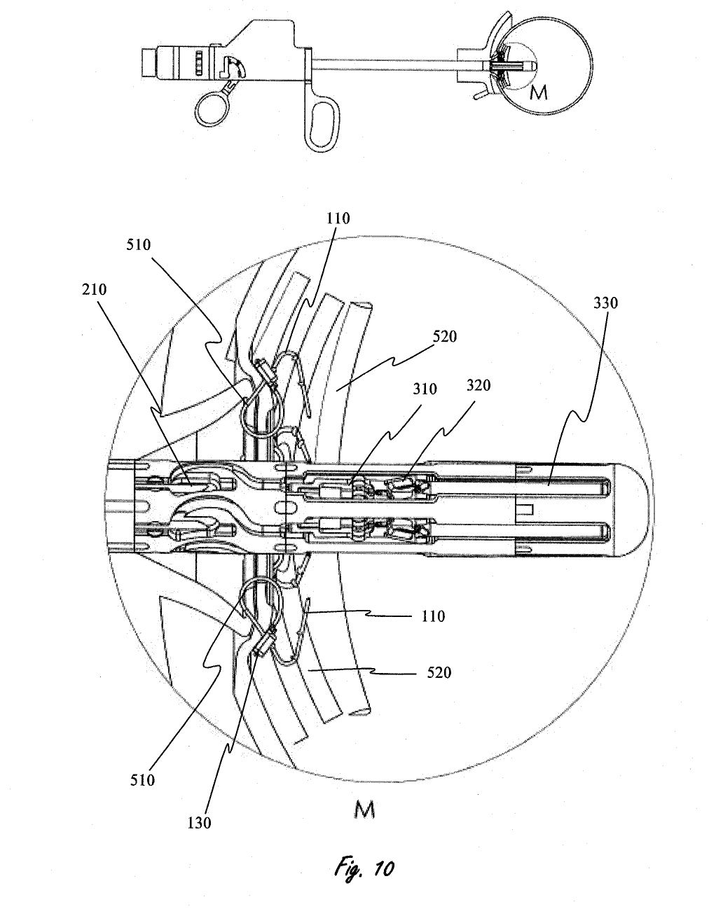

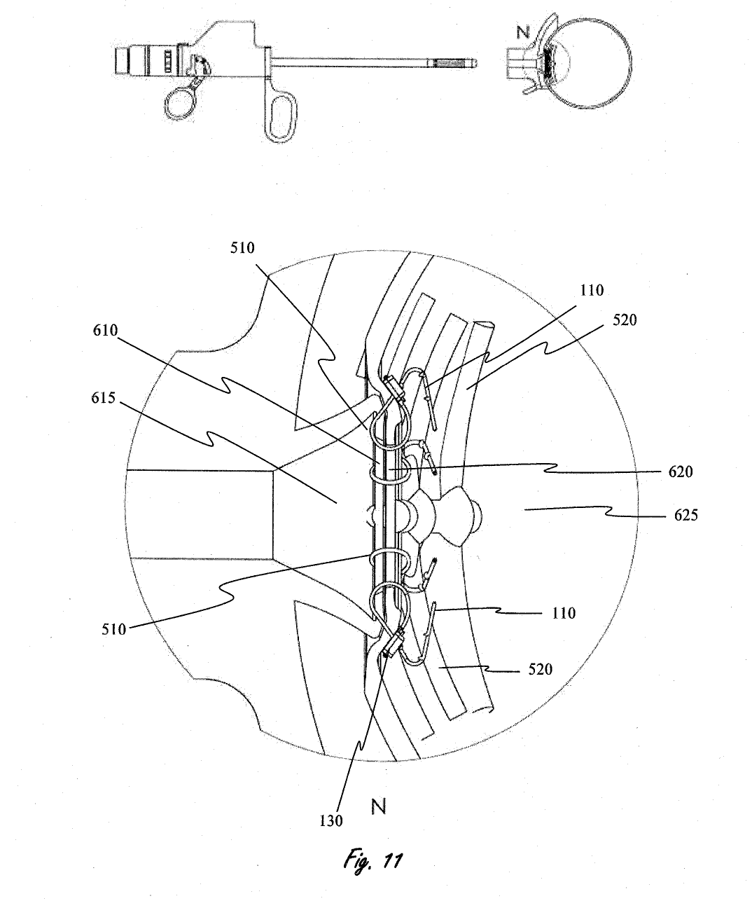

FIGS. 12A and 12B are schematic cut-views of the first and second hollow organs [610,620], e.g. urethra and bladder neck, demonstrating the resulted tied-sutures [510].

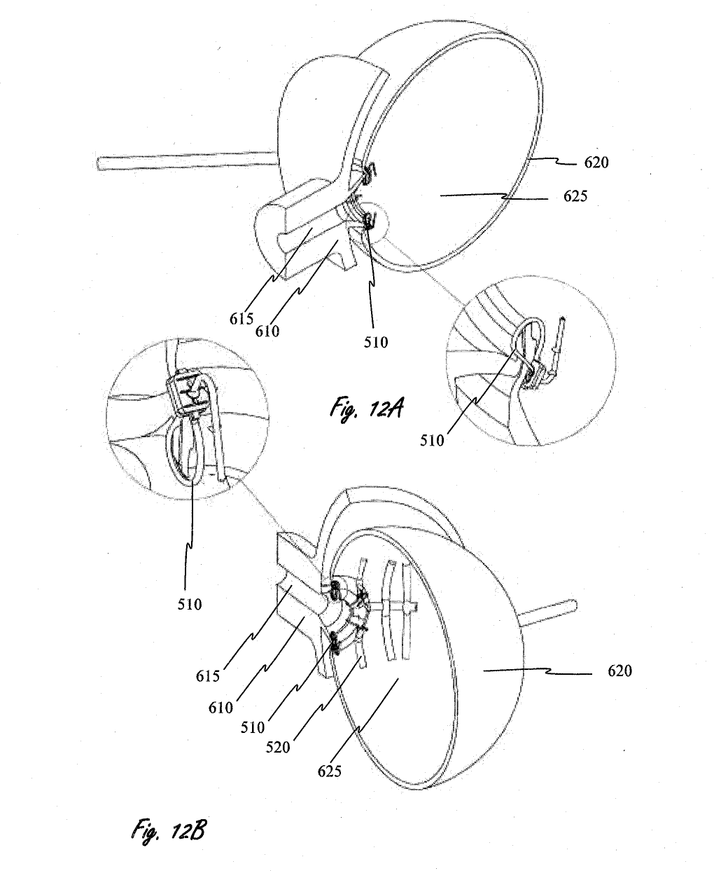

Reference is now made to FIG. 13 which is a schematic cut-view of the device's longitudinal section [340], disclosing the proximal guiding element [410] and the distal guiding element [420] configured for the guidance of the guiding-shaft [210]. According to an embodiment of the present invention, the distal guiding-element [420] is controlled by a horizontal slide [425] moving forward and backward, such that the distal guiding-element [420] is protruded and retracted accordingly. FIG. 13 further discloses another embodiment of the present invention, where the longitudinal section [340] further comprises at least one proximal guiding-element [410], demonstrated in FIG. 13 as a guiding tunnel, configured to guide the guiding-shaft [210] into the female-head [130], via the tissues of the first and second hollow organs [610,620]. The proximal guiding-element [410], according to another embodiment of the present invention, comprises a shape selected from a group consisting of: U-shape, V-shape, circular-shape, elliptic-shape, polygonal-shape, and any combination thereof.

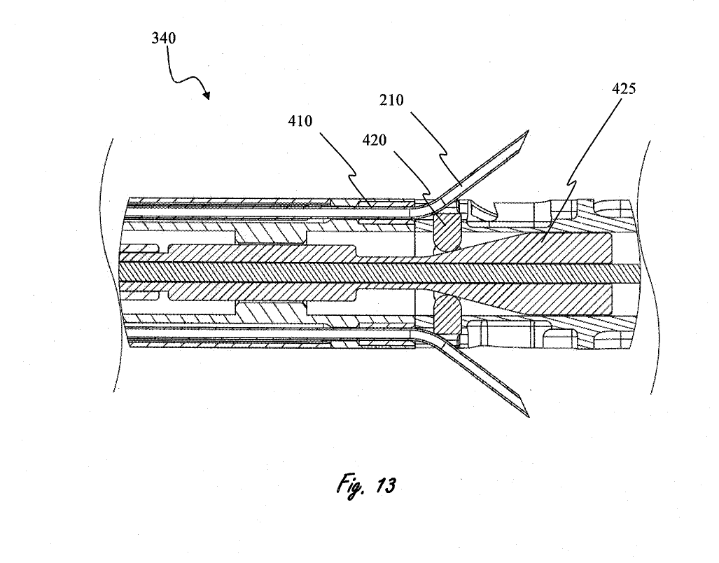

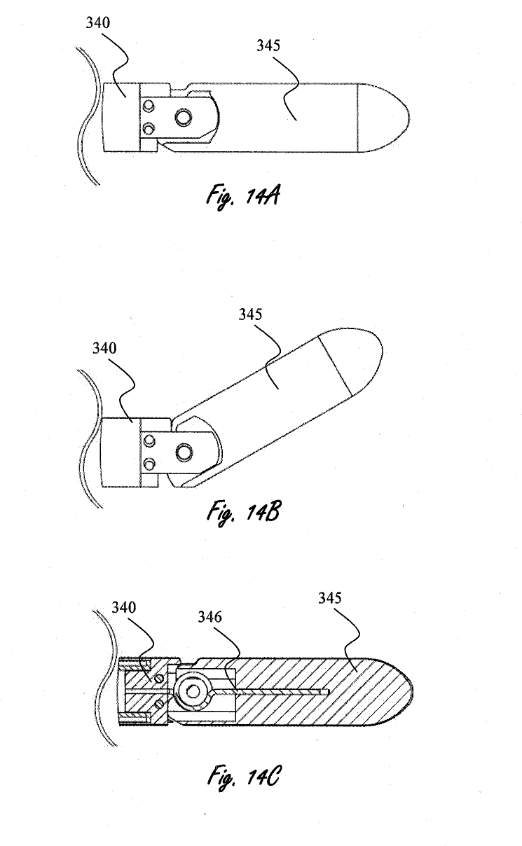

Reference is now made to FIGS. 14A, 14B and 14C, which are schematic drawing disclosing the device's longitudinal section [340] having a flexible head [345]. Two configurations are demonstrated: straiten, as in FIG. 14A, and diverted, as in FIG. 14B. FIG. 14C is a cut view if the flexible head [345] disclosing a spring [346] there within. The spring [346] is configured for allowing the head [345] to be diverted according lumens [615,625] configuration and then to be straiten back.

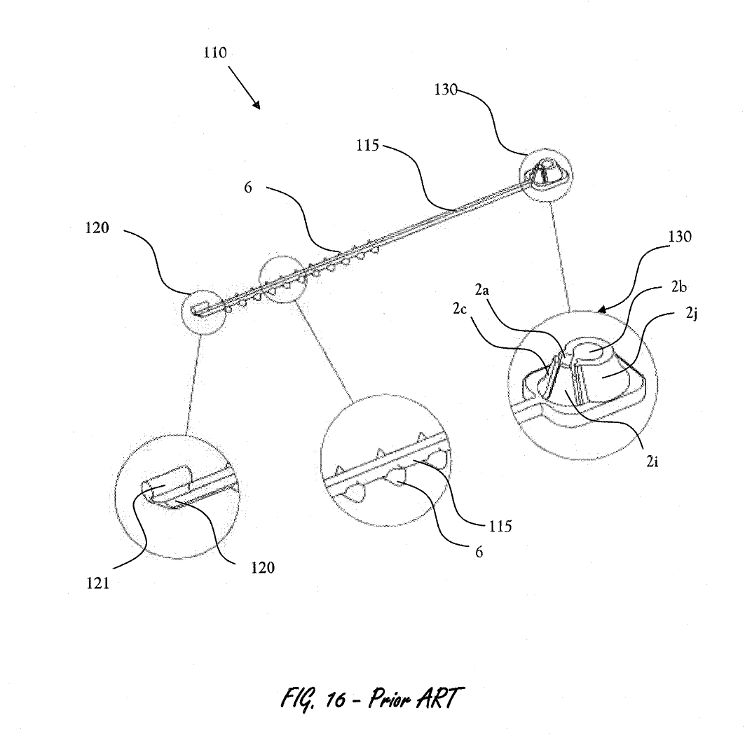

Reference is now made to prior art FIGS. 15 and 16 which are prior art schematic views of the fastenable-suture [110] and the guiding-shaft [210], describing the various components of same. The fastenable-suture [110] comprises an elongated flexible cord [115], with one or more teeth [6] protruding from same, a male-head [120] at the distal end of the cord [115] and an accepting female-head [130] at the proximal end. The male-head [120] is in connection with an anchor [121]. The anchor [121] configured to be temporarily accommodated by a distal end [4b] of the guiding-shaft [210]. The female-head [130] comprising a suture accepting-channel [2a] and a neighboring guiding-shaft accepting-channel [2b]. The suture accepting-channel [2a] comprises one or more flexible pawls [2c] for both allowing, by means of the teeth [6], a continuous and linear forward motion of the male-head [120] and the cord [115] within the suture accepting-channel [2a] in only one direction, while preventing the suture's [110] male-head [120] and cord [115] motion in a backward direction and allowing only the guiding-shaft [210] a reversible reciprocal motion.

Reference is now made to prior art FIGS. 17A, 17B, 17C, 17D and 17E which are schematic drawings of the fastenable-suture [110], comprising the accepting channels [2i, 2j]. FIGS. 17A-17E demonstrate the fastenable-suture [110] being pushed by the guiding-shaft [210], shown in five stages: FIG. 17A demonstrates the approaching distal end [4b] of the guiding-shaft [210] together with the suture's male-head [120] towards the suture's female-head [130]; FIG. 17B demonstrates the distal end [4b] of the guiding-shaft [210] together with the sutures' male-head [120] entering into the suture's female-head [130]; FIG. 17C demonstrates the fastening of the first distal tooth [6]; FIG. 17D demonstrates the retracting of the guiding-shaft [210] back and out of the female-head [130], while the fastenable-suture [110] is fixed by the fastened tooth [6]; and FIG. 17E demonstrates the detaching of the guiding-shaft [210] from the fastenable-suture [110].

FIGS. 17C, 17D and 17E further demonstrate another embodiment for the fastenable-suture [110] where the guiding-shaft [210] further comprises a disengaging-rod [215] threaded through the central axis of the guiding-shaft [210], configured for relative motion inside the guiding-shaft [210] in order to disengage the anchor [121] out of the guiding-shaft's [210] bore [4d] and notch [4g]. When the guiding-shaft [210] is retracting backward, as in FIG. 17D, the disengaging-rod [215] stands still preventing the anchor's [121] movement. When anchor [121] is detached from the distal end [4b] of the guiding-shaft [210] the disengaging-rod [215] retracts out of the female-head [130] joining the retracted guiding-shaft [210], as shown in FIG. 17E. FIG. 17C further demonstrates another embodiment where the anchor [121] comprises a rod-bore [3a] configured to temporarily accommodate the disengaging-rod [215].

FIGS. 17A and 17D further demonstrate other embodiments for the present invention where the guiding-shaft [210] distal head comprises the sharp portion [4c] and where the male-head [120] comprises a sharp-tip [1a], both configured for smooth passage through the tissues of the first and second hollow organs [610,620].

Reference is now made to prior art FIG. 18 which is a schematic drawing of another embodiment for the present invention, where the suture's cord [115] comprises a weakened section [116] which is thinner than the rest of the cord [115], located between the anchor [121] and the first distal tooth [6], configured to be ripped facilitate detachment of the male-head [120] from the cord [115], thereby from the guiding-shaft [120], when the cord's [115] tensile tension has reached a predetermined tension. This configuration is set for avoiding unnecessary or even harmful stress to the first and second hollow organs [610,620]. In a preferred embodiment the thickness of the weakened section [116] is set to be ripped at predetermined tension of about 400 gram-force.

1-41. (canceled)

42. A device [100] for suturing a first hollow organ [610] to a second hollow organ [620], comprising:
a. a guiding-shaft [210], configured for temporary attachment to a first end of a fastenable-suture [110] and guidance of the same via the tissue of said first and second hollow organs [610,620]; and
b. a foldable arm [310], configured to accommodate the second end of said fastenable-suture [110] and to receive said guiding-shaft [210] with said first end;
wherein said device [100] configured to manipulate said guiding-shaft [210] and said foldable arm [310] via the lumens [615,625] of said first and second hollow organs [610,620], such that said fastenable-suture [110] is guided and fastened to form a tied-suture [510] in a single act.

43. The device according to claim 42, wherein said device [100] further comprises:
a. at least one additional guiding-shaft [210], for at least one additional fastenable-suture [110]; and
b. at least one additional foldable arm [310];
such that at least two independent tied-sutures [510] are simultaneously formed, from said lumens [615,625] of said first and second hollow organs [610,620], in said single act.

44. The device according to claim 42, wherein said fastenable-suture [110] comprises:
a. a cord [115];
b. a male-head [120] having an anchor [121], at first end of said cord [115], configured for said temporary attachment with said guiding-shaft [210]; and
c. an accepting female-head [130], at opposite end of said cord [115], configured to receive said male-head [120] and form said tied-suture [510].

45. The device according to claim 44, wherein said foldable arm [310] comprises an open-housing [320] configured to temporarily accommodate said female-head and release the same, upon retraction of said foldable arms [310].

46. The device according to claim 43, wherein said device further comprising a longitudinal section [340]; said longitudinal section [340] configured to:
a. accommodate at least one said foldable arm [310];
b. accommodate at least one said guiding-shaft [210]; and
c. temporarily accommodate at least one said fastenable-suture [110].

47. The device according to claim 42, wherein said foldable arm [310] configured to push and approximate said second hollow organ [620] towards said first hollow organ [610].

48. The device according to claims 44 and 46, wherein said longitudinal section further comprises at least one proximal guiding-element [410], configured to guide said guiding-shaft [210] into said female-head [130], via said tissues of said first and second hollow organs [610,620].

49. The device according to claim 48, wherein said proximal guiding-element comprises a shape selected from a group consisting of: U-shape, V-shape, circular-shape, elliptic-shape, polygonal-shape, and any combination thereof.

50. The device according to claim 44, wherein said device [100] further comprises at least one distal guiding-element [420], configured to guide said guiding-shaft [210] into said female-head [130], via said tissues of said first and second hollow organs [610,620].

51. The device according to claim 50, wherein said distal guiding-element [420] is controlled by a horizontal slide [425] moving forward and backward, such that said distal guiding-element [420] is protruded and retracted accordingly.

52. The device according to claim 42, wherein said foldable arm [310] comprises a foldable shield [330] configured to protect said second hollow organ [620] from undesired damage by said guiding-shaft [210];

53. The device according to claim 52, wherein said shield [330] is at least partially flexible.

54. The device according to claim 44, wherein said suture's cord [115] comprises a weakened section [116], which is thinner than said cord [115], configured to be ripped or lengthened to limit over tightening of said tied-suture [510].

55. The device according to claim 44, wherein said guiding-shaft [210] comprises a disengaging-rod [215] threaded through said guiding-shaft [210], configured for relative motion inside said guiding-shaft [210]; said disengaging-rod [215] configured to disengage and detach said male-head [210] from said guiding-shaft [210], such that said anchor [121] of said tied suture [510] released and/or detached from said guiding-shaft [210].

56. The device according to claim 42, wherein said device [100] configured to penetrate into said first and second hollow organ [610,620] with or without puncturing said first and/or second hollow organs [610,620].

57. The device according to claim 42 or 43, wherein said fastenable-suture [110] is guided and fastened to form said tied-suture [510] in at least one additional act.

58. A method for suturing a first hollow organ [610] to a second hollow organ [620], comprising steps of:
a. approximating said first and second hollow organs [610,620] one to another; and
b. suturing said first and second hollow organs [610,620] one to another, by means of forming at least one tied-suture [510];
wherein said steps of approximating and suturing performed via the lumens [615,625] of said first and second hollow organs [610,620].

59. The method according to claim 58, further comprising steps of:
a. penetrating into said first hollow organ [610], via an orifice [614] in said first hollow organ [610] or by puncturing the same;
b. penetrating into said second hollow organ [620], via an orifice [626] in said second hollow organ [620] or by puncturing the same; and
c. attaching said first and second hollow organs [610,620] one to another.

60. The method according to claim 58, further comprising steps:
a. temporary attaching at least one fastenable-suture [110] to at least one guiding-shaft [210];
b. guiding said guiding-shaft [210] via the tissue of said first and second hollow organs [610,620]; and
c. receiving a said guiding-shaft [210] by at least one foldable arm [310];
thereby fastening said fastenable-suture [110] and forming said at least one tied-suture [510] in at least one act.

61. The method according to claim 60, further comprising step of providing said fastenable-suture [110] with:
a. a cord [115];
b. a male-head [120] having an anchor [121], at first end of said cord [115], for said temporarily attaching said guiding-shaft [210]; and
c. an accepting female-head [130], at opposite end of said cord [115], for receiving said male-head [120] and fastening said tied-suture [510].

62. The method according to claim 61, wherein said step of suturing further comprising:
a. guiding said male-head [120] into said tissues of said first and second hollow organs [610,620], by said guiding-shaft [210]; and
b. accepting said male-head [120], by said female-head [130].

63. The method according to claim 60, further comprising step of selecting at least one step from a group consisting of:
a. pushing the walls of said first hollow organ [610], by protruding said guiding-shaft [210];
b. protruding said foldable arm [310];
c. retracting said foldable arm [310];
d. folding said foldable arm [310];
e. pushing said second hollow organ [620] toward said first hollow organ [610];
f. fastening said tied-suture [510];
g. retracting said guiding-shaft [210].

64. The method according to claims 60, further comprising step of selecting at least one step from a group consisting of:
a. disconnecting said female-head [120] from foldable arm [310], upon retraction of said foldable arm [310];
b. disconnecting said male-head [120] from said cord [115]; and
c. any combination thereof.

65. The method according to claim 60, further comprising steps of temporarily accommodating said female-head [130] by an open-housing [320] within said foldable arm [310] and releasing the same, upon retracting of said foldable arm [210].

66. The method according to claim 60, further comprising step of providing at least one proximal guiding-element [410], configured for guiding said guiding-shaft into said female-head [130], via said first and second hollow organs [610,620].

67. The method according to claim 60, further comprising step of providing at least one distal guiding-element [420], configured for guiding said guiding-shaft into said female-head [130], via said first and second hollow organs [610,620].

68. The method according to claim 67, further comprising step of selecting at least one step from a group consisting of controlling said distal guiding-element [420] with a horizontal slide [425], moving forward and backward, thereby protruding and retracting said distal guiding-element [420] accordingly.

69. The method according to claim 59, further comprising step protecting said second hollow organ [620] from undesired damage by said guiding-shaft [210], by providing said foldable arm [310] with a foldable shield [330].

70. The method according to claim 60, further comprising steps ripping and detaching said male-head [120] from said cord [115], when said cord's [115] tensile tension is reaching a predetermined tension force, by providing said suture's cord with a weakened section [116].

71. The method according to claim 60, further comprising steps of disengaging and separating said male-head [120] from said guiding-shaft [210], by providing said guiding-shaft [210] with a disengaging-rod [215] threaded through the center axis of said guiding-shaft [210] and configured for moving inside said guiding-shaft [210], thereby releasing said fastenable-suture [110] from said guiding-shaft [210].

72. A device [100] for suturing a first hollow organ [610] to a second hollow organ [620], comprising:
a. a guiding-shaft [210], configured for temporary attachment to a first end of a fastenable-suture [110] and guidance of the same via the tissue of said first and second hollow organs [610,620]; and
b. an arm, configured to accommodate the second end of said fastenable-suture and to receive said guiding-shaft [210] with said first end;
wherein said device [100] configured to manipulate said guiding-shaft [210] and said arm via the lumens [615,625] of said first and second hollow organs [610,620], such that said fastenable-suture [110] is guided and fastened to form a tied-suture [510] in a single act.

73. The device according to claim 72, wherein said device [100] further comprises:
a. at least one additional guiding-shaft [210], for at least one additional fastenable-suture [110]; and
b. at least one additional arm;
such that at least two independent tied-sutures [510] are simultaneously formed, from said lumens [615,625] of said first and second hollow organs [610,620], in said single act.
